(19) United States
(12) Patent Application Publication
(10) Pub. No.: US 2015/0347716 A1
(43) Pub. Date: Dec. 3, 2015

(54) Method for Supervising Consumption of a Medical Food for Prevention and/or Management of a Disease or Condition

(71) Applicant: PHOTONZ CORPORATION, Auckland (NZ)

(72) Inventor: Gregory Paul Moss-Smith, Red Hill (AU)

(73) Assignee: Photonz Corporation Ltd., Auckland (NZ)

(21) Appl. No.: 14/378,712

(22) PCT Filed: Feb. 14, 2013
(86) PCT No.: PCT/NZ2013/000013

§ 371 Date: Aug. 14, 2014

(30) Foreign Application Priority Data
Feb. 14, 2012 (NZ) 598218

(51) Int. Cl. G06F 19/00; G01N 33/66; G01N 33/92; G01N 33/49

(52) U.S. Cl. CPC G06F 19/3475; G06F 19/322; G01N 33/49; G01N 33/66; G01N 33/92

(57) ABSTRACT

A method for supervising a patient during prevention and/or management of a disease or condition involving a medical food having greater than 60% pure eicosapentaenoic acid.

### TECHNICAL FIELD

The present disclosure relates to a method for supervising a patient during prevention and/or management of a disease or condition involving a medical food having greater than 60% pure eicosapentaenoic acid.

### BACKGROUND

It was discovered in the late 1920s that certain “essential” dietary fatty acids must be present in effective quantities for normal growth and health in rats (Burr & Burr J. Biol. Chem., 82: 345-367 1929). Epidemiological data collected from human populations beginning in the 1940s then suggested relatively high dietary intakes of omega-3 Highly Unsaturated Fatty Acids (n-3 HUFA) may be protective against the development of a number of health and medical conditions and that diet deficient in n-3 HUFA intake has an adverse impact on health and may increase the risk of, for example, cardiovascular disease (Sinclair. Lancet 1:381-3 1956; Bang et al., Lancet 1:1143-5.1971; Hirai et al., Lancet 2:1132-3. 1980; Kromhout et al Am J. Clin. Nutr., 85:1142-1147).

In recent decades supplementation studies incorporating individual n-3 HUFA in the diet of humans have demonstrated beneficial health effects of addressing the nutritional deficiency of individual dietary n-3 HUFA. In particular, human dietary supplementation studies incorporating relatively pure forms of the n-3 HUFA eicosapentaenoic acid (EPA) have suggested this nutrient may promote health and ameliorate or even reverse the effects of a range of common diseases and conditions, including but not limited to certain forms of cardiovascular disease and depression (Yokoyama et al., Lancet 369:1062-1063. 2007; Peet & Horrobin Arch. Gen. Psych. 59(10) 913-9 2002).

Cardiovascular disease, in particular, is one of the major causes of death in the United States and most European countries. It is estimated that approximately 70 million people in the United States alone suffer from a cardiovascular disease or disorders including, but not limited to, high blood pressure, coronary heart disease, dyslipidemia, hyperlipidemia, congestive heart failure and stroke.

Online digital technologies are commonly used to communicate product details to healthcare practitioners and supervisors about pharmaceuticals and medical foods, including n-3 HUFA, and are rapidly replacing traditional pharmaceutical and medical food sales representatives and common differential marketing messages about treatments of diseases, including cardiovascular diseases. Crowd sourcing information and other technologies that build professional networks and provide a two-way dialogue allow customers and patients to share their thoughts with other patients and doctors in real time. Pharmaceutical and medical food marketing has concentrated on providing static information to physicians and patients. For example, a website might communicate information to a user about a given product including drug facts, clinical study results, interactions, side effects, etc. This information conveys important data to a patient or physician. However, the path for this information is only in one direction: from the pharmaceutical or medical food producer to the patient or physician.

There is a need in the prior art for a system to allow for an online two-way exchange of information regarding medical foods comprising n-3 HUFA. There is also a need in the art for an online system to allow for the exchange of information from the medical food supplier to the patient, physician or medical supervisor about the consumption of a medical food comprising n-3 HUFA. There is also a need in the art for an online system to allow for the exchange of information from the patient, physician or medical supervisor back to the medical food supplier. There is also a need in the art for an online system to allow for the exchange of information from the patient to the physician or medical supervisor. There is a need in the art for an online system to provide a technological framework for an interactive method to supervise a patient's consumption of a medical food in order to market the medical food to both the patient and physician.

The invention has overcome some or all of the deficiencies in the prior art.

### SUMMARY

In a first aspect, the invention is a method for supervising the consumption of a medical food in a patient, wherein the medical food comprises EPA in any biologically assimilable form, the method comprising: analyzing a blood sample using a frontend computer to determine the cholesterol, diglyceride and/or triglyceride levels in the blood sample; receiving patient registration data and a measure of the cholesterol, diglyceride and/or triglyceride levels in the blood sample corresponding to the patient at a backend computer via a network connection; extracting, with the backend computer, nutrition management data for the patient from the patient registration data and the blood data; determining, with the backend computer, dosage data from the nutrition management data, the dosage data including an amount of the medical food to manage a disease or condition for the patient; and sending the dosage data from the backend computer to a frontend computer via the network connection. Preferably, the EPA is at least 50% pure. More preferably, the EPA is at least 60% pure. Even more preferably, the EPA is at least 70% pure. Even more preferably, the EPA is at least 80% pure.

In a second aspect, the invention is a method for supervising the consumption of a medical food in a patient, wherein the medical food comprises EPA in any biologically assimilable form, the method comprising: receiving patient registration data corresponding to the patient and a measure of cholesterol, diglyceride and/or triglyceride levels from a blood sample from the patient at a backend computer via a network connection; displaying, with the backend computer, nutrition management data for the patient from the patient registration data and the blood data; determining dosage data from the nutrition management data, the dosage data including an amount of the medical food to manage a disease or condition for the patient; and sending the dosage data from the backend computer to a frontend computer via the network connection. Preferably, the EPA is at least 50% pure. More preferably, the EPA is at least 60% pure. Even more preferably, the EPA is at least 70% pure. Even more preferably, the EPA is at least 80% pure.

Preferably, the medical food is used to manage and/or prevent the disease or condition by modulating a specific surrogate marker of the disease or condition.

Preferably, the disease or condition is selected from the group consisting of; (1) any psychiatric, neurological or other central or peripheral nervous system disease; in particular schizophrenia, depression, bipolar disorder and degenerative disorders of the brain including Alzheimer's disease and other dementias and Parkinson's disease; (2) asthma and other respiratory diseases; (3) diseases of the gastrointestinal tract including inflammatory bowel diseases and irritable bowel syndrome; inflammatory disease affecting any system; (4) cardiovascular disease including mixed dyslipidemia, hyperlipidemia and hypertriglyceridemia; (6) any form of diabetes or any form of metabolic diseases; (7) dermatological diseases; (8) kidney or urinary tract diseases; (9) liver diseases; (10) disease of the male or female reproductive organs such as the breast or the prostate gland; (11) cancer or cancer cachexia; (12) diseases of the head and neck, including disease of the mouth and teeth, of the eyes or of the ears; and (13) infection with viruses, bacteria, fungi, protozoa or other micro-organisms.

More preferably, the disease or condition is cardiovascular disease. Even more preferably, the cardiovascular disease is selected from the group consisting of: mixed dyslipidemia, hyperlipidemia and hypertriglyceridemia.

Preferably, the patient has one or more of: a baseline triglyceride level of about 300 mg/dl to about 1500 mg/dl; baseline non-HDL-C value of about 200 mg/dl to about 400 mg/dl; baseline total cholesterol value of about 250 mg/dl to about 400 mg/dl; baseline vLDL-C value of about 140 mg/dl to about 200 mg/dl; baseline HDL-C value of about 10 to about 60 mg/dl; and/or baseline LDL-C value of about 50 to about 300 mg/dl.

Preferably, the patient has one or more of: a baseline triglyceride level of about 150 mg/dl to about 1500 mg/dl, a baseline non-HDL-C value of about 200 mg/dl to about 300 mg/dl; a baseline total cholesterol value of about 250 mg/dl to about 300 mg/dl; a baseline vLDL-C value of about 140 mg/dl to about 200 mg/dl; and/or a baseline HDL-C value of about 10 to about 80 mg/dl.

Preferably, the patient has one or more of: a baseline triglyceride level (or median baseline triglyceride level in the case of a subject group), fed or fasting, of at least about 300 mg/dl; at least about 400 mg/dl; at least about 500 mg/dl; at least about 600 mg/dl; at least about 700 mg/dl; at least about 800 mg/dl; at least about 900 mg/dl; at least about 1000 mg/dl; at least about 1100 mg/dl; at least about 1200 mg/dl; at least about 1300 mg/dl; at least about 1400 mg/dl; or at least about 1500 mg/dl.

Preferably, the patient has one or more of: a fasting baseline absolute plasma level of EPA (or mean thereof in the case of a subject group) not greater than about 0.70 nmol/ml (about 0.21 μg/ml); not greater than about 0.65 nmol/ml (about 0.20 μg/ml); not greater than about 0.60 nmol/ml (about 0.18 μg/ml); not greater than about 0.55 nmol/ml (about 0.17 μg/ml); not greater than about 0.50 nmol/ml (about 0.15 μg/ml); not greater than about 0.45 nmol/ml (about 0.14 μg/ml); or not greater than about 0.40 nmol/ml (about 0.12 μg/ml).

Preferably, the patient has one or more of: a baseline fasting plasma level (or mean thereof) of EPA, expressed as a percentage of total free fatty acid, of not more than about 3%; not more than about 2.5%; not more than about 2%; not more than about 1.5%; not more than about 1%; not more than about 0.75%; not more than about 0.5%; not more than about 0.25%, not more than about 0.2% or not more than about 0.15%.

Preferably, the patient has one or more of: a fasting baseline absolute plasma level of total fatty acid (or mean thereof) not greater than about 250 nmol/ml; not greater than about 200 nmol/ml; not greater than about 150 nmol/ml; not greater than about 100 nmol/ml; or not greater than about 50 nmol/ml.

Preferably, the patient has one or more of: a fasting baseline plasma, serum or red blood cell membrane EPA level not greater than about 231 nmol/ml (about 70 μg/ml); not greater than about 198 nmol/ml (about 60 μg/ml); not greater than about 165 nmol/ml (about 50 μg/ml); not greater than about 132 nmol/ml (about 40 μg/ml); not greater than about 99 nmol/ml (about 30 μg/ml); or not greater than about 83 nmol/ml (about 25 μg/ml).

Preferably, the medical food comprises EPA in any biologically assimilable form, selected from the group consisting: at least 50% pure EPA in any biologically assimilable form; at least 60% pure EPA in any biologically assimilable form; at least 65% pure EPA in any biologically assimilable form; at least 70% pure EPA in any biologically assimilable form; at least 75% pure EPA in any biologically assimilable form; at least 80% pure EPA in any biologically assimilable form; at least 85% pure EPA in any biologically assimilable form; at least 90% pure EPA in any biologically assimilable form; at least 95% pure EPA in any biologically assimilable form; at least 96% pure EPA; at least 97% pure EPA in any biologically assimilable form; at least 98% pure EPA; at least 99% pure EPA in any biologically assimilable form.

Preferably, the medical food comprises docosahexaenoic acid selected from the group consisting of: less than 5% docosahexaenoic acid; less than 3% docosahexaenoic acid; less than 1% docosahexaenoic acid; and no detectable levels of docosahexaenoic acid.

Preferably, the medical food comprises arachidonic acid in any biologically assimilable form, selected from the group consisting of: less than 5% arachidonic acid in any biologically assimilable form; less than 3% arachidonic acid in any biologically assimilable form; less than 1% arachidonic acid in any biologically assimilable form; and no detectable levels of arachidonic acid in any biologically assimilable form.

Preferably, medical food is selected from the group consisting of; (1) at least 90% pure EPA in any biologically assimilable form, 5% or less than 5% of arachidonic acid in any biologically assimilable form and less than 1% docosahexaenoic acid in any biologically assimilable form; and (2) at least 95% pure EPA in any biologically assimilable form, 3% or less than 3% of arachidonic acid in any biologically assimilable form and less than 1% docosahexaenoic acid in any biologically assimilable form.

Preferably, the medical food is comprised in a capsule, flavoured oil blend, emulsifier or other liquid form, microencapsulated powder or other dry form vehicle. More preferably, the medical food is comprised in a capsule.

In one embodiment, the medical food is not nonconcentrated fish oil. In another embodiment, the medical food is not unenriched fish oil. In another embodiment, the medical food is not a composition that comprises less than 30% pure EPA in any biologically assimilable form. In another embodiment, the medical food is not a composition that comprises less than 40% pure EPA in any biologically assimilable form. In another embodiment, the medical food is not a composition that comprises less than 50% pure EPA in any biologically assimilable form. In another embodiment, the medical food is not a composition that comprises less than 60% pure EPA in any biologically assimilable form.

Preferably, the amount of the medical food to manage a disease or condition comprises between 1 g to 4 g of at least 60% pure EPA in any biologically assimilable form.

Preferably, the dosage data includes instructions that the medical food is consumed by the patient 1 to 4 times per day.

Preferably, the dosage data includes instructions that the medical food is consumed by the patient over a period of about 1 to about 500 weeks, about 1 to about 200 weeks, about 1 to about 100 weeks, about 1 to about 80 weeks, about 1 to about 50 weeks, about 1 to about 40 weeks, about 1 to about 20 weeks, about 1 to about 15 weeks, about 1 to about 12 weeks, about 1 to about 10 weeks, about 1 to about 5 weeks, about 1 to about 2 weeks or about 1 week.

Preferably, the patient consumes the medical food for about 12 weeks, the patient exhibits one or more of (a) reduced triglyceride levels compared to baseline; (b) reduced Apo B levels compared to baseline; (c) increased HDL-C levels compared to baseline; (d) a reduction in non-HDL-C levels compared to baseline; and/or (e) a reduction in vLDL levels compared to baseline.

Preferably, the patient exhibits one or more of: (a) a reduction in triglyceride level of at least about 5% as compared to baseline; (b) a less than 30% increase in non-HDL-C levels or a reduction in non-HDL-C levels of at least about 1% as compared to baseline; (c) an increase in HDL-C levels of at least about 5% as compared to baseline; and/or (d) a less than 60% in LDL-C levels compared to baseline.

Preferably, the patient exhibits one or more of: (a) a reduction in triglyceride level of at least about 30% as compared to baseline; (b) no increase in non-HDL-C levels as compared to baseline; (c) no decrease in HDL-C levels compared to baseline; and/or (d) a less than 30% increase in LDL-C levels as compared to baseline.

Preferably, the patient consumes the medical food, the patient exhibits one or more of: (a) reduced triglyceride levels compared to baseline; (b) reduced Apo B levels compared to baseline; (c) increased HDL-C levels compared to baseline; (d) no increase in LDL-C levels compared to baseline; (e) a reduction in LDL-C levels compared to baseline; (f) a reduction in non-HDL-C levels compared to baseline; (g) a reduction in vLDL levels compared to baseline; (h) an increase in apo A-I levels compared to baseline; (i) an increase in apo A-I/apo B ratio compared to baseline; (j) a reduction in lipoprotein a levels compared to baseline; (k) a reduction in LDL particle number compared to baseline; (1) an increase in LDL size compared to baseline; (m) a reduction in remnant-like particle cholesterol compared to baseline; (n) a reduction in oxidized LDL compared to baseline; (o) a less than 5% change in fasting plasma glucose (FPG) compared to baseline; (p) a less than 5% change in haemoglobin Alc (HbAic) compared to baseline; (q) a reduction in homeostasis model insulin resistance compared to baseline; (r) a reduction in lipoprotein associated phospholipase A2 compared to baseline; (s) a reduction in intracellular adhesion molecule compared to baseline; (t) a reduction in interleukin-6 compared to baseline; (u) a reduction in plasminogen activator inhibitor compared to baseline; (v) a reduction in high sensitivity C-reactive protein (hsCRP) compared to baseline; (w) an increase in serum phospholipid EPA compared to baseline; and/or (x) an increase in red blood cell membrane EPA compared to baseline.

Preferably, the dosage data includes instructions that the medical food is consumed to manage a cardiovascular disease or condition or surrogate marker thereof.

Preferably, the medical food is packaged in blister packages of about 1 to about 20 capsules per sheet.

Preferably, the front end computer then contacts a dispensary of the medical food and arranges shipment of the medical food in the established dose to the specific patient. More preferably, the patient receives the medical food by delivery to their residence. Alternatively, the patient is notified (for example, by email or text message from the front end computer) where they can pick up the medical food. In one example, as soon as shipment has been arranged, the patient is notified by the front end computer (by email or text for example) when the expected delivery date is-estimated to occur.

In a third aspect, the invention is a computing device for supervising the consumption of a medical food in a patient, wherein the medical food comprises >60% pure EPA in any biologically assimilable form, the method comprising: a network interface coupled to a communication network; a processor coupled to the network interface; a memory coupled to the processor; and a medical supervisor application stored in the memory, wherein the application is configured to execute on the processor to: receive, at the computer, patient registration data and blood data from a remote computer via the communications network interface; extract, at the computer, nutrition management data from the patient registration data; and display, at the computer, the nutrition management data to determine dosage data from the nutrition management data, the dosage data including an amount of the medical food to manage a disease or condition for the patient.

In a further aspect, the invention is a computing system for supervising the consumption of a medical food in a patient with dyslipidemia via a network wherein the medical food comprises >60% pure EPA in any biologically assimilable form, the method comprising: a client computing device including a processor, and a memory storing first instructions to cause the processor to send patient registration data via the network; a server computing device including a processor, an interface with the network, and a memory storing second instructions to cause the processor to: receive the patient registration data and blood data from the client computing device via the communications network interface; extract nutrition management data from the patient registration data; and display nutrition management data to determine dosage data from the nutrition management data, the dosage data including an amount of the medical food to manage a disease or condition for the patient; and send the dosage data to the to the client computing device via the network.

The features and advantages described in this summary and the following detailed description are not all-inclusive. Many additional features and advantages will be apparent to one of ordinary skill in the art in view of the drawings, specification, and claims hereof.

### BRIEF DESCRIPTION OF THE DRAWINGS

FIG. 1 illustrates a block diagram of an exemplary medical food consumption supervising system in accordance with the described embodiments;

FIG. 2 illustrates an exemplary block diagram of a method for using the medical food consumption supervising system in accordance with the described embodiments;

FIG. 3 illustrates a high-level block diagram of a computing environment that implements modules of the medical food consumption supervising system in accordance with the described embodiments.

The figures depict a preferred embodiment of the present invention for purposes of illustration only. One skilled in the art will readily recognize from the following discussion that alternative embodiments of the structures and methods illustrated herein may be employed without departing from the principles of the invention described herein.

### DETAILED DESCRIPTION

Those skilled in the art will appreciate that the invention described herein is susceptible to variations and modifications other than those specifically described. It is to be understood that the invention includes all such variations and modifications. The invention also includes all of the steps, features, compositions and materials referred to or indicated in the specification, individually or collectively and any and all combinations or any two or more of the steps or features.

The present invention is not to be limited in scope by the specific embodiments described herein, which are intended for the purpose of exemplification only. Functionally equivalent products, compositions and methods are clearly within the scope of the invention as described herein.

The invention described herein may include one or more ranges of values (e.g. size, concentration, etc). A range of values will be understood to include all values within the range, including the values defining the range, and values adjacent to the range that lead to the same or substantially the same outcome as the values immediately adjacent to that value which defines the boundary to the range.

The entire disclosures of all publications (including patents, patent applications, journal articles, laboratory manuals, books, or other documents) cited herein are hereby incorporated by reference. Inclusion does not constitute an admission is made that any of the references constitute prior art or are part of the common general knowledge of those working in the field to which this invention relates.

Throughout this specification, unless the context requires otherwise, the word “comprise” or variations, such as “comprises” or “comprising” will be understood to imply the inclusion of a stated integer, or group of integers, but not the exclusion of any other integers or group of integers. It is also noted that in this disclosure, and particularly in the claims and/or paragraphs, terms such as “comprises”, “comprised”, “comprising” and the like can have the meaning attributed to it in US patent law; e.g. they can mean “includes”, “included”, “including”, and the like.

“Medical food” as used herein includes medical foods defined in section 5(b) of the US Orphan Drug Act (21 U.S.C. 360ee (b) (3)) as “a food which is formulated to be consumed or administered enterally under the supervision of a physician and which is intended for the specific dietary management of a disease or condition for which distinctive nutritional requirements, based on recognized scientific principles, are established by medical evaluation.” Preferably, the term “medical food” includes a composition that satisfies the US Food and Drug Agency's regulations at 21 CFR 101.9(j) (8), that is, a food is a medical food exempt from nutrition labelling only if:
(a) It is a specially formulated and processed product (as opposed to a naturally occurring foodstuff used in its natural state) for the partial or exclusive feeding of a patient by means of oral intake or enteral feeding by tube;
(b) It is intended for the dietary management of a patient who, because of therapeutic or chronic medical needs, has limited or impaired capacity to ingest, digest, absorb, or metabolize ordinary foodstuffs or certain nutrients, or who has other special medically determined nutrient requirements, the dietary management of which cannot be achieved by the modification of the normal diet alone;
(c) It provides nutritional support specifically modified for the management of the unique nutrient needs that result from the specific disease or condition, as determined by medical evaluation;
(d) It is intended to be used under medical supervision; and
(e) It is intended only for a patient receiving active and ongoing medical supervision wherein the patient requires medical care on a recurring basis for, among other things, instructions on the use of the medical food.

Medical foods are intended for the specific clinical dietary management of a disease or condition for which distinctive nutritional requirements are established by medical evaluation based on recognized scientific principles. Medical foods may be a therapeutic adjunct to diet to reduce elevated triglyceride levels in patients with hypertriglyceridemia and at high risk of cardiovascular disease. The medical food which is the subject of this invention may reduce triglyceride levels and other important lipid and inflammation biomarkers, including Apo-B, non-HDL-C, Total-Cholesterol, VLDL-C, Lp-PLA2, and hs-CRP without increasing LDL-C. Other omega-3 fatty acid and fibrate therapies that lower triglycerides have been shown to increase levels of LDL-C.

“Medical supervisor” as used herein includes a person or medical professional registered or authorized to administer a medical food to a patient, including a physician or nurse.

“Patient registration data” as used herein includes information about the patient's age, sex, weight, relative level of fitness and health, history of illness, family history of illness, and current prescribed pharmaceutical medications and medical foods and dosages.

“Blood data” as used herein includes levels of cholesterol, diglycerides and triglycerides amongst other parameters described herein. Blood data parameters can be measured in accordance with any clinically acceptable methodology. For example, triglycerides, total cholesterol, HDL-C and fasting blood sugar can be sampled from serum and analyzed using standard photometry techniques. VLDL-TG, LDL-C and VLDL-C can be calculated or determined using serum lipoprotein fractionation by preparative ultracentrifugation and subsequent quantitative analysis by refractometry or by analytic ultracentrifugal methodology. Apo Al, Apo B and hsCRP can be determined from serum using standard nephelometry techniques. Lipoprotein (a) can be determined from serum using standard turbidimetric immunoassay techniques. LDL particle number and particle size can be determined using nuclear magnetic resonance (NMR) spectrometry. Remnants lipoproteins and LDL-phospholipase A2 can be determined from EDTA plasma or serum and serum, respectively, using enzymatic immunoseparation techniques. Oxidized LDL, intercellular adhesion molecule-1 and interleukin-6 levels can be determined from serum using standard enzyme immunoassay techniques. These techniques are described in detail in standard textbooks, for example Tietz Fundamentals of Clinical Chemistry, 6^{th} Ed. (Burtis, Ashwood and Borter Eds.), WB Saunders Company. In one embodiment, subjects fast for up to 12 hours prior to blood sample collection, for example about 10 hours.

“Nutrition management data” as used herein includes information about the particular nutritional requirements of the patient needed to prevent or manage a disease or medical condition. The nutrition management data may be in the form of a written report, either in paper form or in electronic form. Preferably the nutrition management data is in electronic form and contains a report on whether the patient needs to modify his or her diet and whether they are to consume a medical food to prevent or manage a disease or medical condition.

“Dosage data” as used herein includes the quantity and frequency of the consumption of the medical food by the patient. Preferably, the medical food is consumed in an effective amount.

“Effective amount” as used herein with respect to methods of prevention and management and, in particular, dosage, shall mean that dosage that provides the specific response for which the medical food is consumed in a significant number of subjects in need of such management or prevention. It is emphasized that an “effective amount,” administered to a particular subject in a particular instance will not always be effective in managing or preventing the diseases or condition described herein, even though such dosage is deemed an “effective amount” by those skilled in the art. It is to be further understood that dosages are, in particular instances, measured as oral dosages, or with reference to levels as measured in blood.

The term “management” in relation a given disease or disorder, includes, but is not limited to, inhibiting the disease or condition, for example, arresting the development of the disease or condition; relieving the disease or condition, for example, causing regression of the disease or condition; relieving a condition caused by or resulting from the disease, for example, relieving, preventing or treating symptoms of the disease or disorder, or influencing in a positive sense a surrogate marker of the disease condition, for example, the reduction of level of a marker that is known to be positively correlated with a particular disease.

The term “prevention” in relation to a given disease or condition means: preventing the onset of disease or condition development if none had occurred, preventing the disease or condition from occurring in a subject that may be predisposed to the disease or condition but has not yet been diagnosed as having the disorder or disease, and/or preventing further disease/condition development if already present.

The term “cardiovascular disease” herein refers to any disease or disorder of the heart or blood vessels (i.e. arteries and veins) or any symptom thereof. Non-limiting examples of cardiovascular-related disease and disorders include hyperlipidemia, hypertriglyceridemia, hypercholesterolemia, mixed dyslipidemia, coronary heart disease, vascular disease, stroke, atherosclerosis, arrhythmia, hypertension, myocardial infarction, and other cardiovascular events.

“Mixed dyslipidemia” refers to a condition in which patients have a combination of two or more lipid abnormalities in their blood including elevated triglycerides, diglycerides, low high-density lipoprotein cholesterol (HDL-C), and elevated low-density lipoprotein cholesterol (LDL-C).

“Hyperlipidemia” as used herein is characterized as the abnormal elevation of lipids in the blood.

“Hypertriglyceridemia” as used herein is characterized as the abnormal elevation of tryglycerides in the blood. Elevated triglycerides are clinically stratified into three groups: very high triglycerides (>500 mg/dL), high triglycerides (>200 and <500 mg/dL) and borderline high triglycerides (>150 and <200 mg/dL).

“Eicosapentaenoic acid” (or “EPA”) as used herein is well known in the art and in the physiological literature it is given the name 20:5(n-3). The IUPAC name for eicosapentaenoic acid is (5Z,8Z,11Z,14Z,17Z)-eicosa-5,8,11,14,17-pentaenoic acid. Methods to manufacture medical foods comprising eicosapentaenoic acid or esterified derivatives of EPA are well known in the art including methods to purify eicosapentaenoic acid or its esters from fish oil and algae. Eicosapentaenoic acid is available in a number of assimilable forms, including ethyl eicosapentaenoate or eicosapentaenoyl-ethyl ester (EPA-EE), as well as in esters including glycerol such as a triglyceride, diglyceride, or monoglyceride, or in the free fatty acid form. Highly pure EPA-EE (>95%) formulated as a medical food has potential benefits of preventing and treating hypertriglyceridemia and mixed dyslipidemia (multiple lipid disorders), especially in patients with very high triglyceride levels (≧500 mg/dL) and patients with high triglyceride levels (≧200 and <500 mg/dL) who are also on statin therapy for elevated LDL-cholesterol levels. Significant scientific and clinical evidence support the efficacy and safety of EPA-EE in reducing triglyceride levels and other important lipid and inflammation biomarkers, including Apo-B, Total-Cholesterol, VLDL-C, Lp-PLA2, and hs-CRP without increasing LDL-C.

“Arachidonic acid” as used herein is well known in the art and in the physiological literature it is given the name 20:4(w-6). The IUPAC name for arachidonic acid is (5Z,8Z,11Z,14Z)-5,8,11,14-Eicosatetraenoic acid. Methods to manufacture medical foods comprising arachidonic acid are well known in the art including methods to purify arachidonic acid from fish oil, fungi, and algae. Arachidonic acid is available in a number of assimilable forms, including arachidonyl-ethyl ester (ARA-EE).

“Docosahexaenoic acid” as used herein is well known in the art and in the physiological literature it is given the name 22:6(n-3). The IUPAC name for docosahexaenoic acid is (4Z,7Z,10Z,13Z,16Z,19Z)-docosa-4,7,10,13,16,19-hexaenoic acid. Docosahexaenoic acid is available in a number of assimilable forms, including docosahexaenoyl-ethyl ester (DHA-EE).

### EXAMPLES

### Example 1

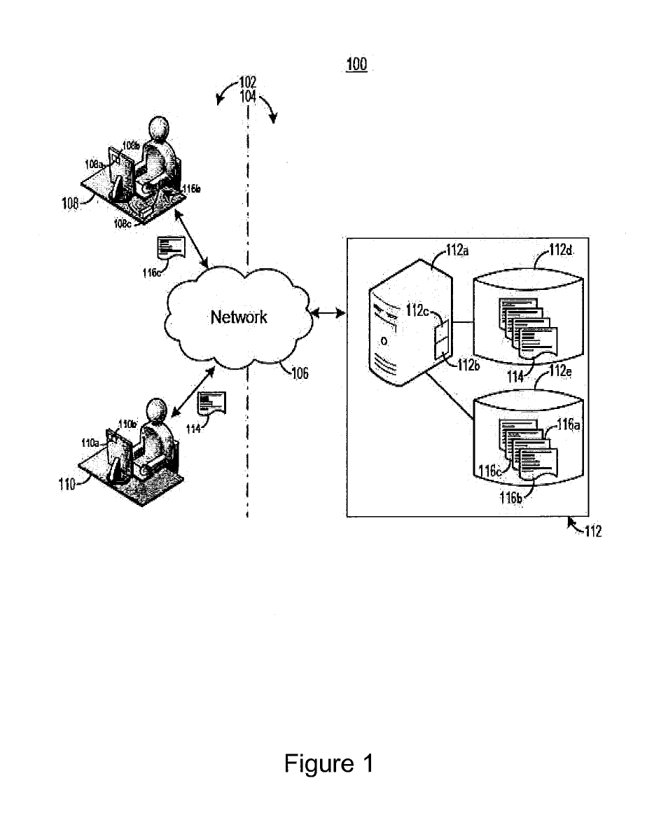

### FIG. 1 Description

FIG. 1 illustrates a block diagram of a high-level architecture of a medical food consumption supervising system 100 including various software and hardware components or modules that may be employed to monitor a patient's course of management with the medical food.

The system 100 may include front end components 102 and back end components 104 that communicate with each other via a network 106. Front end components 102 generally include computing devices that face a user but may also include telephonic networks and devices. In some embodiments, the front end devices 102 include a patient computing device 108 and a physician or other medical supervisor computing device 110. For example the computing devices 108, 110 may include a Wi-Fi or web-enabled personal computer, cellular phone, tablet computer, or other personal computing device capable of wireless or wired communication between the front end components 102 and back end components 104. Both the patient and physician computing devices may include processors 108a, 110a and memories 108b, 110b, respectively. Each of the memories 108b, 110b may store instructions for execution by their respective processors 108a, 110a for supervising the consumption of a medical food during a course of management prescribed by a physician using the physician computing device 110. The various instructions may be stored in the memories as modules or functions. In some embodiments, the memories 108b, 110b and modules or functions may be implemented as computer-readable storage memories containing computer-readable instructions (i.e. software) for execution by a local or remote processor of a front end or back end computer system. The modules may perform the various tasks associated with supervising management of hypertriglyceridemia with a medical food, where the medical food has greater than 60% eicosapentaenoic acid in an assimilable form, as herein described. The system 100 also includes both hardware and software applications, as well as various data communications channels for communicating data between the various hardware and software components.

A physician may use the physician computing device 110 to prescribe a medical food to a patient. In some embodiments, the patient may be undergoing treatment for dyslipidemia or hypertriglyceridemia and require a prescription for one or more drugs to lower cholesterol levels. For example, the patient may require a prescription for a drug having greater than 60% EPA-EE for the treatment of hypertriglyceridemia, as herein described.

The physician may use the computing device 110 to submit a prescription 114 for a medical food to a backend server system 112. The backend server system includes a server computing device 112a having both a processor 112b and memory 112c. Upon receiving a prescription 114 via the network 106, the processor 112c of the server system 112 may execute instructions from the memory 112c to store the prescription 114 within a local or remote database 112d as a text file or other type of file. In some embodiments, the database 112d is a component of the server 112a or another computing device. Once the data 114 is received and stored, the patient computing device 108 may access the prescription data 114 via the network 106 and pull the data 114 to the device 108 for viewing or other processing. In further embodiments, the server system 112 may push or send the prescription data 114 entered by the physician to the patient computing device 108 via the network 106 (e.g. by e-mail, SMS message, or other method).

The patient computing device 108 may also include a monitoring device 108c. The monitoring device 108c may include a processor and a memory, storing instructions that may be executed by the processor. In some embodiments, instructions stored in the memory of the monitoring device 108 analyze physical characteristics 116a of the patient and send an analysis result 116b to the server system 112 via the network 106. For example, physical characteristics may include blood measurements (cholesterol, diglyceride and/or triglyceride levels, etc), cardio respiratory characteristics (pulse, blood pressure, VO2 levels, etc.), weight, body composition (BMI, fat or lean mass, etc.), etc. Upon receiving the analysis result 116b via the network 106, the processor 112c of the server system 112 may execute instructions from the memory 112c to store the result data 116b within a local or remote database 112d as a text file or other type of file. The results data 116b may then be pulled or pushed to the physician computing device 110 to be read by the physician. In further embodiments, instructions stored in the memory of the monitoring device 108 measure physical characteristics 116a of the patient and send the characteristics 116a to the server system 112 via the network 106. Upon receiving the measured characteristics 116a via the network 106, the processor 112c of the server system 112 may execute instructions from the memory 112c to analyze the characteristics 116a and store the analysis result data 116b within a local or remote database 112d as a text file or other type of file. The either or both of the characteristics data 116a or results data 116b may then be pulled or pushed to the physician computing device 110 to be read by the physician at the physician device 110. Where the characteristics data 116a is sent to the physician device 110, instructions stored in the memory 110b of the physician device 110 may be executed to analyze the received data 116a to determine an analysis result 116b. In further embodiments, the computer-readable memory at any of the patient computing device 108, the monitoring device 108c, the server system 112, and the physician device 110 may include instructions to analyze the patient characteristics 116a. The memory of the monitoring device 108 may include instructions to receive and analyze patient characteristics 116a related to blood, other bodily fluids, or any other physical characteristics of the patient. For example, when the patient is being managed for dyslipidemia, the monitoring device 108 (or patient computing device, server system 112, physician computing device, etc.) may include instructions to measure and/or analyze cholesterol, diglyceride and triglyceride levels in a blood sample of the patient that are executed on any processor of the system 100. In still further embodiments, the physician computing device 110 may receive the patient characteristic data 116a and a human physician may analyze the data 116a to produce analysis results 116b. Analysis data may also be input directly by a human physician at the computing device 110 and transferred to the server system 112 via the network 106.

The analysis results 116b may include instructions for a patient's course of management regarding consumption of a medical food. For example, where the characteristics data 116a indicates the patient should consume more or less of a medical food that was prescribed for a course of management, then the analysis results 116b may include instructions for the patient to modify his or her behavior accordingly. In a further example, the course of management may include management of dyslipidemia or hypertriglyceridemia and the medical food may be a drug having greater than 60% EPA-EE. Where the characteristics data indicates an unhealthy level of cholesterol, diglycerides and/or triglycerides (e.g. a triglyceride level at or above 200 mg/dL or a triglyceride level at or above 500 mg/dL, etc.), then the analysis results 116b may include an adjustment in a dosage level for the medical food.

In further embodiments, the course of management may be modified or informed by patient input data 116c. For example, a patient may collect and input physical characteristics or the monitoring device 108 may be configured to collect and transmit patient physical characteristics (e.g. weight management, exercise program, diet, prescription compliance, symptoms such as shortness of breath, etc.) to the server system 112 via the network 106. The patient input data 116c may also include periodic patient input that can be reviewed by a physician or the physician computing device 110 to integrate with lab data and potentially modulate the patient prescription data 114.

### Example 2

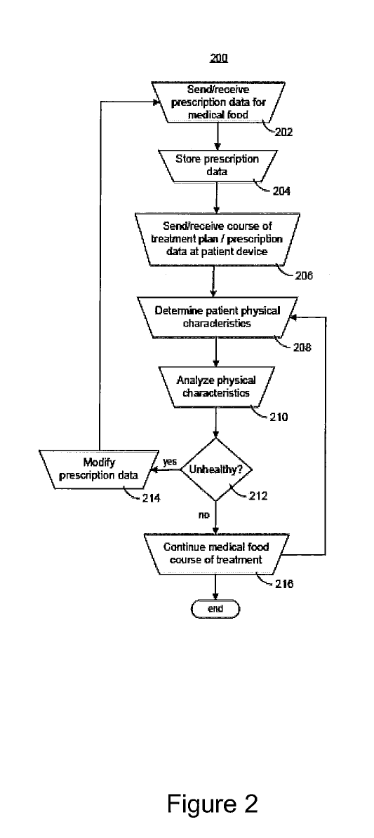

### FIG. 2 Description

With reference to FIG. 2, the medical food consumption supervising system 100 described herein may be employed in a method 200 (FIG. 2) to supervise a patient's consumption of a medical food during a physician-prescribed course of management using the medical food. The method 200 may also market the medical food to both the patient and physician. In particular, the medical food may include a prescription-grade omega-3 fatty acid medical food containing >90% EPA-EE and no DHA-EE. The medical food may be used by patients with dyslipidemia, generally, and hypertriglyceridemia, in particular (i.e. elevated blood levels of triglycerides) who are at high risk of cardiovascular disease and may be taking statins for high cholesterol. The method 200 may include one or more functions or routines in the form of computer-executable instructions that are stored in a tangible computer-readable medium and executed using a processor of a computing device (e.g. the patient computing device 108, the server system 112, the physician computing device 110, etc.). The routines may be included as part of any of the modules described in relation to FIG. 1, or as part of a module that is external to the system illustrated by FIG. 1.

At function 202, the system 100 may execute instructions to receive a prescription 114 for a course of management using a medical food. The prescription 114 may be sent from a physician computing device 110 to the server system 112 via the network 106. For example, a physician may examine a patient and determine that the patient should consume a medical food. Where the examination determines that the patient suffers from dyslipidemia and could benefit from consumption of a medical food having greater than 60% EPA-EE for the management of hypertriglyceridemia, then the prescription data 114 may include such information. In some embodiments, the prescription data 114 may also include a course of management plan including instructions that, when executed by the patient computing device 108, allow the patient to follow the prescribed course of management using the medical food.

At function 204, the system 100 may execute instructions to store the prescription data 114. In some embodiments, the server system 112 may receive the data 114 and store it in a local or remote data repository 112d.

At function 206, the system 100 may execute instructions to send or receive the prescription data/prescription plan 114 at the patient computing device 108. In some embodiments, the server system 112 may send the data 114 to the patient device 108 via the network 106 and the device 108 may display the data 114 on a monitor or other display device which may be portable or permanently fixed.

At function 208, the system 100 may execute instructions to determine patient physical characteristics data 116 and/or management compliance related to the prescribed course of management. In some embodiments, a test may be administered to a patient under a physician's supervision and the test data may be sent to a laboratory for analysis. Analysis results may be sent to the physician computing device 110 via the network 106 for further analysis. In further embodiments, a monitoring device 108c may measure various physical characteristics of the patient. For example, where the monitoring device 108c is able to measure the composition of blood and the patient is being managed for dyslipidemia, then the monitoring device 108c may determine the amount of cholesterol, diglycerides and/or triglycerides in a sample of the patient's blood. In all embodiments, patient physical characteristic data 116 may be collected and analyzed in a clinically acceptable and effective manner.

At function 210, the system 100 may execute instructions to analyze the physical characteristics 116 that were measured at function 208. In some embodiments, the function 210 may refer to the patient's course of management and prescription data 114 to determine baseline levels of the physical characteristics data 116. For example, an initial examination of the patient may reveal a triglyceride level at or above 200 mg/dL in a blood analysis of the patient. After consuming a medical food having greater than 60% EPA-EE for a period of time, the function 210 may analyze the physical characteristics to determine that the patient's triglyceride level has decreased or increased from the initial baseline examination.

At function 212, if the physical characteristics data 116 analyzed at function 210 indicates an unhealthy condition that may be managed by the medical food, then the system 100 may execute instructions to modify the prescription data 114 at function 214 and return to function 202. Alternatively, the physical characteristics data 116 analyzed at function 210 may not indicate an unhealthy condition that may be managed by the medical food. If the unhealthy condition does not exist, the system 100 may execute instructions to return to function 208. After another period of time, the patient may used the monitoring device 108c to determine another set of physical characteristics 116 to further determine of the prescription for the medical food should be modified.

As described herein, a communication system may provide a technological framework for an interactive method to supervise a patient's consumption of a medical food. Using network-enabled computing devices, a patient may be monitored remotely for adherence to a course of management that uses the medical food. In some embodiments, a physician may receive periodic measurements or analysis of the patient's physical characteristics related to the medical food and the course of management to determine if the prescription for the medical food should be modified. In particular, the medical food may include a prescription-grade omega-3 fatty acid medical food containing >90% EPA-EE and no DHA. The medical food may be used by patients with hypertriglyceridemia (i.e. elevated blood levels of triglycerides) who are at high risk of cardiovascular disease and may be taking statins for high cholesterol. The interaction between the patient, physician, and server system may increase the physician's immediate awareness of the efficacy of the medical food and promote the use of that medical food with future patients.

### Example 3

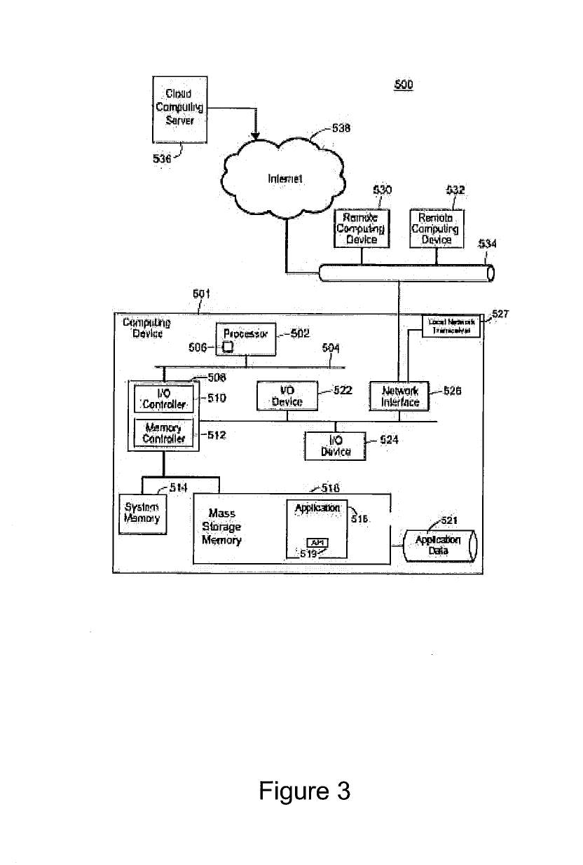

### FIG. 3 Description

FIG. 3 is a high-level block diagram of an example computing environment for a system to remotely supervise the consumption of a medical food in a patient with dyslipidemia via a network having a computing device 501 that may be used to implement the method and systems described herein. The computing device 501 may include a client computing device (e.g. a cellular phone, a tablet computer, a Wi-Fi-enabled device or other personal computing device capable of wireless or wired communication), a thin client, or other known type of computing device. As will be recognized by one skilled in the art, in light of the disclosure and teachings herein, other types of computing devices can be used that have different architectures. Processor systems similar or identical to the example remote medical food consumption supervision system 500 may be used to implement and execute the example system of FIG. 1, the method of FIG. 2, and the like. Although the example system 500 is described below as including a plurality of peripherals, interfaces, chips, memories, etc., one or more of those elements may be omitted from other example processor systems used to implement and execute the example system 100 to remotely supervise the consumption of a medical food in a patient with dyslipidemia. Also, other components may be added.

As shown in FIG. 3, the computing device 501 includes a processor 502 that is coupled to an interconnection bus 504. The processor 502 includes a register set or register space 506, which is depicted in FIG. 3 as being entirely on-chip, but which could alternatively be located entirely or partially off-chip and directly coupled to the processor 502 via dedicated electrical connections and/or via the interconnection bus 504. The processor 502 may be any suitable processor, processing unit or microprocessor. Although not shown in FIG. 3, the computing device 501 may be a multi-processor device and, thus, may include one or more additional processors that are identical or similar to the processor 502 and that are communicatively coupled to the interconnection bus 504.

The processor 502 of FIG. 3 is coupled to a chipset 508, which includes a memory controller 510 and a peripheral input/output (I/O) controller 512. As is well known, a chipset typically provides I/O and memory management functions as well as a plurality of general purpose and/or special purpose registers, timers, etc. that are accessible or used by one or more processors coupled to the chipset 508. The memory controller 510 performs functions that enable the processor 502 (or processors if there are multiple processors) to access a system memory 514 and a mass storage memory 516.

The system memory 514 may include any desired type of volatile and/or non-volatile memory such as, for example, static random access memory (SRAM), dynamic random access memory (DRAM), flash memory, read-only memory (ROM), etc. The mass storage memory 516 may include any desired type of mass storage device. For example, if the computing device 501 is used to implement an application to remotely supervise the consumption of a medical food in a patient with dyslipidemia 518 having an API 519 (including instructions as described by the method 200 of FIG. 2), the mass storage memory 516 may include a hard disk drive, an optical drive, a tape storage device, a solid-state memory (e.g. a flash memory, a RAM memory, etc.), a magnetic memory (e.g. a hard drive), or any other memory suitable for mass storage. As used herein, the terms module, block, function, operation, procedure, routine, step, and method refer to tangible computer program logic or tangible computer executable instructions that provide the specified functionality to the computing device 501 and the system 500. Thus, a module, block, function, operation, procedure, routine, step, and method can be implemented in hardware, firmware, and/or software. In one embodiment, program modules and routines are stored in mass storage memory 516, loaded into system memory 514, and executed by a processor 502 or can be provided from computer program products that are stored in tangible computer-readable storage mediums (e.g. RAM, hard disk, optical/magnetic media, etc.). Mass storage 516 may also include a cache memory 521 storing application data, user profile data, and timestamp data corresponding to the application data, and other data for use by the application 518.

The peripheral I/O controller 510 performs functions that enable the processor 502 to communicate with peripheral input/output (I/O) devices 522 and 524, and a network interface 526 via a peripheral I/O bus 528. The I/O devices 522 and 524 may be any desired type of I/O device such as, for example, a keyboard, a display (e.g. a liquid crystal display (LCD), a cathode ray tube (CRT) display, etc.), a navigation device (e.g. a mouse, a trackball, a capacitive touch pad, a joystick, etc.), etc. The I/O devices 522 and 524 may be used with the application 518 send the data to the backend components of the system 100, render, and display application data as described in relation to the figures. A local network transceiver 527 may include support for a Wi-Fi network, Bluetooth, Infrared, or other wireless data transmission protocols. In other embodiments, one element may simultaneously support each of the various wireless protocols employed by the computing device 501. For example, a software-defined radio may be able to support multiple protocols via downloadable instructions. In operation, the computing device 501 may be able to periodically poll for visible wireless network transmitters (both cellular and local network) on a periodic basis. Such polling may be possible even while normal wireless traffic is being supported on the computing device 501. The network interface 526 may be, for example, an Ethernet device, an asynchronous transfer mode (ATM) device, an 802.11 wireless interface device, a DSL modem, a cable modem, a cellular modem, etc., that enables the system 100 to communicate with another computer system having at least the elements described in relation to the system 100.

While the memory controller 512 and the I/O controller 510 are depicted in FIG. 3 as separate functional blocks within the chipset 508, the functions performed by these blocks may be integrated within a single integrated circuit or may be implemented using two or more separate integrated circuits. The system 500 may also implement the application 518 on remote computing devices 530 and 532. The remote computing devices 530 and 532 may communicate with the computing device 501 over a network link 534. For example, the computing device 501 may receive patient data created by an application executing on a remote computing device 530, 532. In some embodiments, the application 518 may be retrieved by the computing device 501 from a cloud computing server 536 via the Internet 538. When using the cloud computing server 536, the retrieved application 518 may be programmatically linked with the computing device 501. The application 518 may be a Java® applet executing within a Java® Virtual Machine (JVM) environment resident in the computing device 501 or the remote computing devices 530, 532. The application 518 may also be “plug-ins” adapted to execute in a web-browser located on the computing device 501. In some embodiments, the application 518 may communicate with back end components such as the server 110, etc., via the Internet 538 or other type of network.

Using the systems and procedures described above, the system to remotely supervise the consumption of a medical food in a patient with dyslipidemia via a network 100, 500 may include but is not limited to any combination of a LAN, a MAN, a WAN, a mobile, a wired or wireless network, a private network, or a virtual private network. Moreover, while only three client computing devices 501, 530 and 532 are illustrated in FIG. 3 to simplify and clarify the description, it is understood that any number of client computers are supported and can be in communication within the system 100, 500.

### Example 4

This is a further example of one particular embodiment of practicing the invention. The results presented in this example are expected to be generated.

A patient is concerned about maintaining good cardiovascular health and advises a medical professional of this desire. The patient may be risk of developing or be diagnosed of having mixed dyslipidemia, hyperlipidemia and hypertriglyceridemia.

The medical professional sets up a laboratory in accordance with clinically acceptable methodologies to determine the patient's serum lipids including tryglycerides, total cholesterol, HDL cholesterol, LDL cholesterol and fasting blood sugar. The patient may have one or more of: a baseline triglyceride level of about 300 mg/dl to about 1500 mg/dl; baseline non-HDL-C value of about 200 mg/dl to about 400 mg/dl; baseline total cholesterol value of about 250 mg/dl to about 400 mg/dl; baseline vLDL-C value of about 140 mg/dl to about 200 mg/dl; baseline HDL-C value of about 10 to about 60 mg/dl; and/or baseline LDL-C value of about 50 to about 300 mg/dl. This data is generated and the metrics are entered onto a front end computer.

Additional patient registration data including age, sex, weight, relative level of fitness and health, history of illness, family history of illness, and current prescribed pharmaceutical medications and medical foods and dosages, are also accumulated as a data set on a back end computer. The medical professional queries the backend computer about the patient's laboratory data and lifestyle metrics and this back end computer calculates the dose of the EPA-ethyl ester to be provided to the patient as a medical food for a certain period of time based on the analyzed metrics in order for that patient's serum lipids to be returned to normal levels with a low risk of cardiovascular disease. In one example, the back end computer calculates that the patient is to receive between 1 to 4 g of at least 60% pure EPA-ethyl ester between at least 1 to 4 times per day.

The front end computer then contacts a dispensary of the medical food and arranges shipment of the medical food in the established dose to the specific patient. The patient either receives the medical food by delivery to their residence or is notified (for example by email) where they can acquire the medical food.

After consumption of the medical food for the specified period of time, the patient receives another laboratory test of their serum lipids and the data is analyzed for changes from the original baseline on the back end computer. Based on those changes, the front end computer establishes a new dose and informs the dispensary to release a new product dose package for the patient. The process is repeated until the patient's serum lipids are in an acceptable range and that dose or EPA-ethyl ester is then maintained for that specific patient. In one example, the patient exhibits one or more of: (a) reduced triglyceride levels compared to baseline; (b) reduced Apo B levels compared to baseline; (c) increased HDL-C levels compared to baseline; (d) a reduction in non-HDL-C levels compared to baseline; and/or (e) a reduction in vLDL levels compared to baseline.

Additionally, certain embodiments are described herein as including logic or a number of components, modules, or mechanisms. Modules may constitute either software modules (e.g. code or instructions embodied on a machine-readable medium or in a transmission signal, wherein the code is executed by a processor) or hardware modules. A hardware module is tangible unit capable of performing certain operations and may be configured or arranged in a certain manner. In example embodiments, one or more computer systems (e.g. a standalone, client or server computer system) or one or more hardware modules of a computer system (e.g. a processor or a group of processors) may be configured by software (e.g. an application or application portion) as a hardware module that operates to perform certain operations as described herein.

In various embodiments, a hardware module may be implemented mechanically or electronically. For example, a hardware module may comprise dedicated circuitry or logic that is permanently configured (e.g. as a special-purpose processor, such as a field programmable gate array (FPGA) or an application-specific integrated circuit (ASIC)) to perform certain operations. A hardware module may also comprise programmable logic or circuitry (e.g. as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g. configured by software) may be driven by cost and time considerations.

Accordingly, the term “hardware module” should be understood to encompass a tangible entity, be that an entity that is physically constructed, permanently configured (e.g. hardwired), or temporarily configured (e.g. programmed) to operate in a certain manner or to perform certain operations described herein. As used herein, “hardware-implemented module” refers to a hardware module. Considering embodiments in which hardware modules are temporarily configured (e.g. programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g. over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate on a resource (e.g. a collection of information).

The various operations of example methods described herein may be performed, at least partially, by one or more processors that are temporarily configured (e.g. by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or processors or processor-implemented hardware modules. The performance of certain of the operations may be distributed among the one or more processors, not only residing within a single machine, but deployed across a number of machines. In some example embodiments, the processor or processors may be located in a single location (e.g. within a smart phone, a home environment, an office environment or as a server farm), while in other embodiments the processors may be distributed across a number of locations.

The one or more processors may also operate to support performance of the relevant operations in a “cloud computing” environment or as a “software as a service” (SaaS). For example, at least some of the operations may be performed by a group of computers (as examples of machines including processors), these operations being accessible via a network (e.g. the Internet) and via one or more appropriate interfaces (e.g. application program interfaces (APIs)).

The performance of certain of the operations may be distributed among the one or more processors, not only residing within a single machine, but deployed across a number of machines. In some example embodiments, the one or more processors or processor-implemented modules may be located in a single geographic location (e.g. within a home environment, an office environment, or a server farm). In other example embodiments, the one or more processors or processor-implemented modules may be distributed across a number of geographic locations.

Some portions of this specification are presented in terms of methods, algorithms or symbolic representations of operations on data stored as bits or binary digital signals within a machine memory (e.g. a computer memory). These methods, algorithms or symbolic representations are examples of techniques used by those of ordinary skill in the data processing arts to convey the substance of their work to others skilled in the art. As used herein, an “algorithm” is a self-consistent sequence of operations or similar processing leading to a desired result. In this context, algorithms and operations involve physical manipulation of physical quantities. Typically, but not necessarily, such quantities may take the form of electrical, magnetic, or optical signals capable of being stored, accessed, transferred, combined, compared, or otherwise manipulated by a machine. It is convenient at times, principally for reasons of common usage, to refer to such signals using words such as “data,” “content,” “bits,” “values,” “elements,” “symbols,” “characters,” “terms,” “numbers,” “numerals,” or the like. These words, however, are merely convenient labels and are to be associated with appropriate physical quantities.

Unless specifically stated otherwise, discussions herein using words such as “processing,” “computing,” “calculating,” “determining,” “presenting,” “displaying,” or the like may refer to actions or processes of a machine (e.g. a computer) that manipulates or transforms data represented as physical (e.g. electronic, magnetic, or optical) quantities within one or more memories (e.g. volatile memory, non-volatile memory, or a combination thereof), registers, or other machine components that receive, store, transmit, or display information.

As used herein any reference to “some embodiments” or “an embodiment” means that a particular element, feature, structure, or characteristic described in connection with the embodiment is included in at least one embodiment. The appearances of the phrase “in some embodiments” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

Further, the figures depict preferred embodiments of a system for remotely supervising the consumption of a medical food in a patient with dyslipidemia via a network for purposes of illustration only. One skilled in the art will readily recognize from the following discussion that alternative embodiments of the structures and methods illustrated herein may be employed without departing from the principles described herein. Upon reading this disclosure, those of skill in the art will appreciate still additional alternative structural and functional designs for a system for remotely supervising the consumption of a medical food in a patient with dyslipidemia via a network through the disclosed principles herein. Thus, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

1. A method for supervising the consumption of a medical food in a patient, wherein the medical food comprises at least 60% pure EPA in any biologically assimilable form, the method comprising:
analyzing a blood sample using a frontend computer to determine the cholesterol, diglyceride and/or triglyceride levels in the blood sample;
receiving patient registration data and a measure of the cholesterol, diglyceride and/or triglyceride levels in the blood sample corresponding to the patient at a backend computer via a network connection;
extracting, with the backend computer, nutrition management data for the patient from the patient registration data and the blood data;
determining, with the backend computer, dosage data from the nutrition management data, the dosage data including an amount of the medical food to manage a disease or condition for the patient; and
sending the dosage data from the backend computer to a frontend computer via the network connection.

2. A method for supervising the consumption of a medical food in a patient, wherein the medical food comprises at least 60% pure EPA in any biologically assimilable form, the method comprising:
receiving patient registration data corresponding to the patient and a measure of cholesterol, diglyceride and/or triglyceride levels from a blood sample from the patient at a backend computer via a network connection;
displaying, with the backend computer, nutrition management data for the patient from the patient registration data and the blood data;
determining dosage data from the nutrition management data, the dosage data including an amount of the medical food to manage a disease or condition for the patient; and
sending the dosage data from the backend computer to a frontend computer via the network connection.

3. The method of claims 1 and/or 2, wherein the medical food is used to manage and/or prevent the disease or condition by modulating a specific surrogate marker of the disease or condition.

4. The method of claims 1 to 3, wherein the disease or condition is selected from the group consisting of:
(1) any psychiatric, neurological or other central or peripheral nervous system disease; in particular schizophrenia, depression, bipolar disorder and degenerative disorders of the brain including Alzheimer's disease and other dementias and Parkinson's disease;
(2) asthma and other respiratory diseases;
(3) diseases of the gastrointestinal tract including inflammatory bowel diseases and irritable bowel syndrome; inflammatory disease affecting any system;
(4) cardiovascular disease including mixed dyslipidemia, hyperlipidemia and hypertriglyceridemia;
(6) any form of diabetes or any form of metabolic diseases; (7) dermatological diseases;
(8) kidney or urinary tract diseases;
(9) liver diseases;
(10) disease of the male or female reproductive organs such as the breast or the prostate gland;
(11) cancer or cancer cachexia;
(12) diseases of the head and neck, including disease of the mouth and teeth, of the eyes or of the ears; and
(13) infection with viruses, bacteria, fungi, protozoa or other micro-organisms.

5. The method of claims 1 to 4, wherein the disease or condition is cardiovascular disease.

6. The method of claim 5, wherein the cardiovascular disease is selected from the group consisting of mixed dyslipidemia, hyperlipidemia and hypertriglyceridemia.

7. The method of claims 1 to 6, wherein the patient has one or more of:
a baseline triglyceride level of about 300 mg/dl to about 1500 mg/dl;
baseline non-HDL-C value of about 200 mg/dl to about 400 mg/dl;
baseline total cholesterol value of about 250 mg/dl to about 400 mg/dl;
baseline vLDL-C value of about 140 mg/dl to about 200 mg/dl;
baseline HDL-C value of about 10 to about 60 mg/dl; and/or
baseline LDL-C value of about 50 to about 300 mg/dl.

8. The method of claim 7, wherein the patient has one or more of:
a baseline triglyceride level of about 150 mg/dl to about 1500 mg/dl, a baseline non-HDL-C value of about 200 mg/dl to about 300 mg/dl;
a baseline total cholesterol value of about 250 mg/dl to about 300 mg/dl;
a baseline vLDL-C value of about 140 mg/dl to about 200 mg/dl; and/or
a baseline HDL-C value of about 10 to about 80 mg/dl.

9. The method of claims 1 to 7, wherein the patient has one or more of:
a baseline triglyceride level (or median baseline triglyceride level in the case of a subject group), fed or fasting, of at least about 300 mg/dl;
at least about 400 mg/dl;
at least about 500 mg/dl;
at least about 600 mg/dl;
at least about 700 mg/dl;
at least about 800 mg/dl;
at least about 900 mg/dl;
at least about 1000 mg/dl;
at least about 1100 mg/dl;
at least about 1200 mg/dl;
at least about 1300 mg/dl;
at least about 1400 mg/dl; or
at least about 1500 mg/dl.

10. The method of claims 1 to 9, wherein the patient has one or more of:
a fasting baseline absolute plasma level of EPA (or mean thereof in the case of a subject group) not greater than about 0.70 nmol/ml (about 0.21 μg/ml);
not greater than about 0.65 nmol/ml (about 0.20 μg/ml);
not greater than about 0.60 nmol/ml (about 0.18 μg/ml);
not greater than about 0.55 nmol/ml (about 0.17 μg/ml);
not greater than about 0.50 nmol/ml (about 0.15 μg/ml);
not greater than about 0.45 nmol/ml (about 0.14 μg/ml);
or not greater than about 0.40 nmol/ml (about 0.12 μg/ml).

11. The method of claims 1 to 10, wherein the patient has one or more of:
a baseline fasting plasma level (or mean thereof) of EPA, expressed as a percentage of total free fatty acid, of not more than about 3%;
not more than about 2.5%;
not more than about 2%;
not more than about 1.5%;
not more than about 1%;
not more than about 0.75%;
not more than about 0.5%;
not more than about 0.25%, not more than about 0.2%; or
not more than about 0.15%.

12. The method of claims 1 to 11, wherein the patient has one or more of:
a fasting baseline absolute plasma level of total fatty acid (or mean thereof) not greater than about 250 nmol/ml;
not greater than about 200 nmol/ml;
not greater than about 150 nmol/ml;
not greater than about 100 nmol/ml; or
not greater than about 50 nmol/ml.

13. The method of claims 1 to 12, wherein the patient has one or more of:
a fasting baseline plasma, serum or red blood cell membrane EPA level not greater than about 231 nmol/ml (about 70 μg/ml);
not greater than about 198 nmol/ml (about 60 μg/ml);
not greater than about 165 nmol/ml (about 50 μg/ml);
not greater than about 132 nmol/ml (about 40 μg/ml);
not greater than about 99 nmol/ml (about 30 μg/ml); or
not greater than about 83 nmol/ml (about 25 μg/ml).

14. The method of claims 1 to 13, wherein the medical food comprises EPA in any biologically assimilable form, selected from the group consisting:
at least 65% pure EPA in any biologically assimilable form;
at least 70% pure EPA in any biologically assimilable form;
at least 75% pure EPA in any biologically assimilable form;
at least 80% pure EPA in any biologically assimilable form;
at least 85% pure EPA in any biologically assimilable form;
at least 90% pure EPA in any biologically assimilable form;
at least 95% pure EPA in any biologically assimilable form;
at least 96% pure EPA;
at least 97% pure EPA in any biologically assimilable form;
at least 98% pure EPA;
at least 99% pure EPA in any biologically assimilable form.

15. The method of claims 1 to 14, wherein the medical food comprises docosahexaenoic acid selected from the group consisting of:
less than 5% docosahexaenoic acid;
less than 3% docosahexaenoic acid; and
less than 1% docosahexaenoic acid.

16. The method of claims 1 to 15, wherein the medical food comprises arachidonic acid in any biologically assimilable form, selected from the group consisting of:
less than 5% arachidonic acid in any biologically assimilable form;
less than 3% arachidonic acid in any biologically assimilable form;
less than 1% arachidonic acid in any biologically assimilable form; and
no detectable levels of arachidonic acid in any biologically assimilable form.

17. The method of claims 1 to 16, wherein the medical food is selected from the group consisting of:
at least 90% pure EPA in any biologically assimilable form, 5% or less than 5% of arachidonic acid in any biologically assimilable form and less than 1% docosahexaenoic acid in any biologically assimilable form; and
at least 95% pure EPA in any biologically assimilable form, 3% or less than 3% of arachidonic acid in any biologically assimilable form and less than 1% docosahexaenoic acid in any biologically assimilable form.

18. The method of claims 1 to 17, wherein the medical food is comprised in a capsule, flavoured oil blend, emulsifier or other liquid form, microencapsulated powder or other dry form vehicle.

19. The method of claim 18, wherein the medical food is comprised in a capsule.

20. The method of claims 1 to 19, wherein amount of the medical food to manage a disease or condition comprises between 1 g to 4 g of at least 60% pure EPA in any biologically assimilable form.

21. The method of claims 1 to 20, wherein the dosage data includes instructions that the medical food is consumed by the patient 1 to 4 times per day.

22. The method of claims 1 to 21, wherein the dosage data includes instructions that the medical food is consumed by the patient over a period of about 1 to about 500 weeks, about 1 to about 200 weeks, about 1 to about 100 weeks, about 1 to about 80 weeks, about 1 to about 50 weeks, about 1 to about 40 weeks, about 1 to about 20 weeks, about 1 to about 15 weeks, about 1 to about 12 weeks, about 1 to about 10 weeks, about 1 to about 5 weeks, about 1 to about 2 weeks or about 1 week.

23. The method of claims 1 to 22, wherein after the patient consumes the medical food for about 12 weeks, the patient exhibits one or more of:
(a) reduced triglyceride levels compared to baseline;
(b) reduced Apo B levels compared to baseline;
(c) increased HDL-C levels compared to baseline;
(d) a reduction in non-HDL-C levels compared to baseline; and/or
(e) a reduction in vLDL levels compared to baseline.

24. The method of claim 23, wherein the patient exhibits one or more of:
(a) a reduction in triglyceride level of at least about 5% as compared to baseline;
(b) a less than 30% increase in non-HDL-C levels or a reduction in non-HDL-C levels of at least about 1% as compared to baseline;
(c) an increase in HDL-C levels of at least about 5% as compared to baseline; and/or
(d) a less than 60% in LDL-C levels compared to baseline.

25. The method of claim 23, wherein the patient exhibits one or more of:
(a) a reduction in triglyceride level of at least about 30% as compared to baseline;
(b) no increase in non-HDL-C levels as compared to baseline;
(c) no decrease in HDL-C levels compared to baseline; and/or
(d) a less than 30% increase in LDL-C levels as compared to baseline.

26. The method of claims 1 to 25, wherein after the patient consumes the medical food, the patient exhibits one or more of:
(a) reduced triglyceride levels compared to baseline;
(b) reduced Apo B levels compared to baseline;
(c) increased HDL-C levels compared to baseline;
(d) no increase in LDL-C levels compared to baseline;
(e) a reduction in LDL-C levels compared to baseline;
(f) a reduction in non-HDL-C levels compared to baseline;
(g) a reduction in vLDL levels compared to baseline;
(h) an increase in apo A-I levels compared to baseline;
(i) an increase in apo A-I/apo B ratio compared to baseline;
(j) a reduction in lipoprotein a levels compared to baseline;
(k) a reduction in LDL particle number compared to baseline;
(l) an increase in LDL size compared to baseline;
(m) a reduction in remnant-like particle cholesterol compared to baseline;
(n) a reduction in oxidized LDL compared to baseline;
(o) a less than 5% change in fasting plasma glucose (FPG) compared to baseline;
(p) a less than 5% change in haemoglobin Alc (HbAic) compared to baseline;
(q) a reduction in homeostasis model insulin resistance compared to baseline;
(r) a reduction in lipoprotein associated phospholipase A2 compared to baseline;
(s) a reduction in intracellular adhesion molecule compared to baseline;
(t) a reduction in interleukin-6 compared to baseline;
(u) a reduction in plasminogen activator inhibitor compared to baseline;
(v) a reduction in high sensitivity C-reactive protein (hsCRP) compared to baseline;
(w) an increase in serum phospholipid EPA compared to baseline; and/or
(x) an increase in red blood cell membrane EPA compared to baseline.

27. The method of claims 1 to 26, wherein the dosage data includes instructions that the medical food is consumed to manage a cardiovascular disease or condition or surrogate marker thereof.

28. The method of claims 1 to 27, wherein the medical food is packaged in blister packages of about 1 to about 20 capsules per sheet.

29. A computing device for supervising the consumption of a medical food in a patient, wherein the medical food comprises >60% pure EPA in any biologically assimilable form, the method comprising:
a network interface coupled to a communication network;
a processor coupled to the network interface;
a memory coupled to the processor; and
a medical supervisor application stored in the memory, wherein the application is configured to execute on the processor to:
receive, at the computer, patient registration data and blood data from a remote computer via the communications network interface;
extract, at the computer, nutrition management data from the patient registration data; and
display, at the computer, the nutrition management data to determine dosage data from the nutrition management data, the dosage data including an amount of the medical food to manage a disease or condition for the patient.

30. A computing system for supervising the consumption of a medical food in a patient with dyslipidemia via a network wherein the medical food comprises >60% pure EPA in any biologically assimilable form, the method comprising:
a client computing device including a processor, and a memory storing first instructions to cause the processor to send patient registration data via the network;
a server computing device including a processor, an interface with the network, and a memory storing second instructions to cause the processor to:
receive the patient registration data and blood data from the client computing device via the communications network interface;
extract nutrition management data from the patient registration data; and
display nutrition management data to determine dosage data from the nutrition management data, the dosage data including an amount of the medical food to manage a disease or condition for the patient; and
send the dosage data to the to the client computing device via the network.
